(19) United States
(12) Patent Application Publication
(10) Pub. No.: US 2021/0251677 A1
(43) Pub. Date: Aug. 19, 2021

(54) DEVICES AND METHODS FOR TREATING THE PROSTATE

(71) Applicant: TheraNova, LLC, San Francisco, CA (US)

(72) Inventors: Daniel R. BURNETT, San Francisco, CA (US); Matthew SILVESTRINI, San Francisco, CA (US); Romain ROUX, Vancouver (CA); Michael JAASMA, San Francisco, CA (US)

(73) Assignee: TheraNova, LLC, San Francisco, CA (US)

(21) Appl. No.: 17/240,780

(22) Filed: Apr. 26, 2021

Related U.S. Application Data
(63) Continuation of application No. PCT/US2019/058897, filed on Oct. 30, 2019.
(60) Provisional application No. 62/754,400, filed on Nov. 1, 2018.
(60) Provisional application No. 62/832,705, filed on Apr. 11, 2019.
(60) Provisional application No. 62/874,446, filed on Jul. 15, 2019.

(51) Int. Cl. A61B 18/02

(52) U.S. Cl. CPC A61B 18/02; A61B 2018/00821; A61B 18/0218

(57) ABSTRACT

Devices and methods for treating the prostate are disclosed where in one embodiment, the apparatus generally comprises a catheter which is sized for placement within a body lumen, at least one balloon positioned along a length of the catheter and defining a contact region for thermally contacting a portion of the body lumen which is in proximity to tissue to be treated, and a reservoir in fluid communication with the at least one balloon through at least one lumen defined through the length of the catheter. The at least one balloon may be configured to receive a refrigerant from the reservoir introduced through the at least one lumen until the contact region is at least partially expanded, the catheter being adjustable to selectively align the contact region against the portion of the body lumen to preferentially cool the portion relative to a remainder of the body lumen surrounding the portion.

### CROSS-REFERENCE TO RELATED APPLICATIONS

This application is a continuation of International Patent Application No. PCT/US2019/058897 filed Oct. 30, 2019, which claims the benefit of priority to U.S. Provisional Application No. 62/754,400 filed Nov. 1, 2018, U.S. Provisional Application No. 62/832,705 filed Apr. 11, 2019, and U.S. Provisional Application No. 62/874,446 filed Jul. 15, 2019, each of which is incorporated herein by reference in its entirety.

### FIELD OF THE INVENTION

The present invention relates to treating the prostate.

### INCORPORATION BY REFERENCE

All publications and patent applications mentioned in this specification are herein incorporated by reference to the same extent as if each such individual publication or patent application were specifically and individually indicated to be so incorporated by reference.

### BACKGROUND OF THE INVENTION

The present invention relates to the field of treating benign prostatic hyperplasia and other prostate disease.

Benign prostatic hyperplasia (BPH) arises when the stromal and epithelial cells of the prostate become enlarged. The side effects from this hyperplasia result in the impingement of the urethra. A common method of treatment for benign prostatic hyperplasia (BPH) is transurethral resection of the prostate (TURP) which is the gold standard for surgical treatment. This approach has its disadvantages. The patient often has two recovery days in the hospital and needs to undergo general anesthesia for the operation. There is a need for a minimally invasive treatment of BPH and other prostate diseases, such as cancer.

### SUMMARY OF THE INVENTION

Disclosed herein are embodiments of a transurethral cryotherapy (TC) system and methods of its use. The TC system has several advantages over the gold standard. Since the cryotherapy is less traumatic, the procedure may be performed in an out-patient, or office, setting allowing the patient to go home on the same day. The procedure may also be performed without general anesthesia.

In one embodiment of a transurethral cryotherapy system, the apparatus may generally comprise a catheter which is sized for placement within a body lumen, at least one balloon positioned along a length of the catheter and defining a contact region for thermally contacting a portion of the body lumen which is in proximity to tissue to be treated, and a reservoir in fluid communication with the at least one balloon through at least one lumen defined through the length of the catheter. The at least one balloon may be configured to receive a refrigerant from the reservoir introduced through the at least one lumen until the contact region is at least partially expanded, the catheter being adjustable to selectively align the contact region against the portion of the body lumen to preferentially cool the portion relative to a remainder of the body lumen surrounding the portion.

In another embodiment for a method of applying a cryotherapy treatment, the method may generally comprise advancing a catheter within a body lumen such that at least one balloon positioned along a length of the catheter is placed near or at a portion of the body lumen which is in proximity to tissue to be treated, adjusting a position of the catheter to align a contact region defined along the at least one balloon into contact against the portion of the body lumen, initiating a refrigerant to flow through at least one lumen defined through the length of the catheter and into the at least one balloon such that the contact region is at least partially expanded into thermal contact against the portion of the body lumen, and applying the refrigerant through the contact region until the tissue to be treated is preferentially cooled through the portion of the body lumen relative to a remainder of the body lumen surrounding the portion.

### BRIEF DESCRIPTION OF THE DRAWINGS

FIG. 1 shows an embodiment of the transurethral cryotherapy system.

FIG. 2 shows an embodiment of the TC system in place in a patient.

FIG. 3 shows a cryo-affected zone.

FIG. 4 shows an embodiment of the TC system which includes a heating or insulation area to protect the urethra.

FIG. 5 shows an embodiment of the TC catheter without any insulation.

FIGS. 6 and 7 show embodiments of a TC catheter with variable insulation on the cryotherapy section.

FIG. 8 shows a cryo-affected zone.

FIG. 9 shows a TC catheter with both heating and cooling elements in the cryotherapy section.

FIG. 10 shows the cryotherapy section of FIG. 9 in detail.

FIG. 11 shows a toroidal cryo-affected area.

FIG. 12 shows another embodiment of a TC catheter with both heating and cooling elements.

FIG. 13 shows a cross section of the TC catheter shown in FIG. 12.

FIG. 14 shows an embodiment of the TC catheter including a retention member or balloon and cryotherapy a balloon.

FIG. 15 shows a cross section of the TC catheter shown in FIG. 14

FIG. 16 shows the embodiment of the TC catheter shown in FIG. 14 in place in the urethra.

FIG. 17 shows a possible cross sections of the embodiment shown in FIGS. 14-16.

FIG. 18 shows a possible cross sections of the embodiment shown in FIGS. 14-16.

FIG. 19 shows an embodiment of the TC catheter with multiple balloons radially.

FIG. 20 shows a cross section of an embodiment of the TC catheter with 2 radial balloons. FIG. 18 shows

FIG. 21 shows an embodiment of the TC catheter with multiple balloons radially and longitudinally.

FIGS. 22-29 show cross sections of various TC catheter embodiments.

FIG. 30 shows the prostate anatomy.

FIG. 31 shows an example of components of a controller of the TC System.

FIGS. 32-35 show embodiments of the TC system that allow for directional application of the therapy.

FIGS. 36 and 37 show cross-sectional views of some embodiments of the TC system which incorporate restrictive sheaths.

FIG. 38 shows an embodiment with a sheath that allows for two balloon lobes circumferentially.

FIGS. 39 and 40 show embodiments of the TC system which incorporate one or more needles.

FIG. 41 is a block diagram of a data processing system, which may be used with any embodiments of the invention.

### DETAILED DESCRIPTION OF THE INVENTION

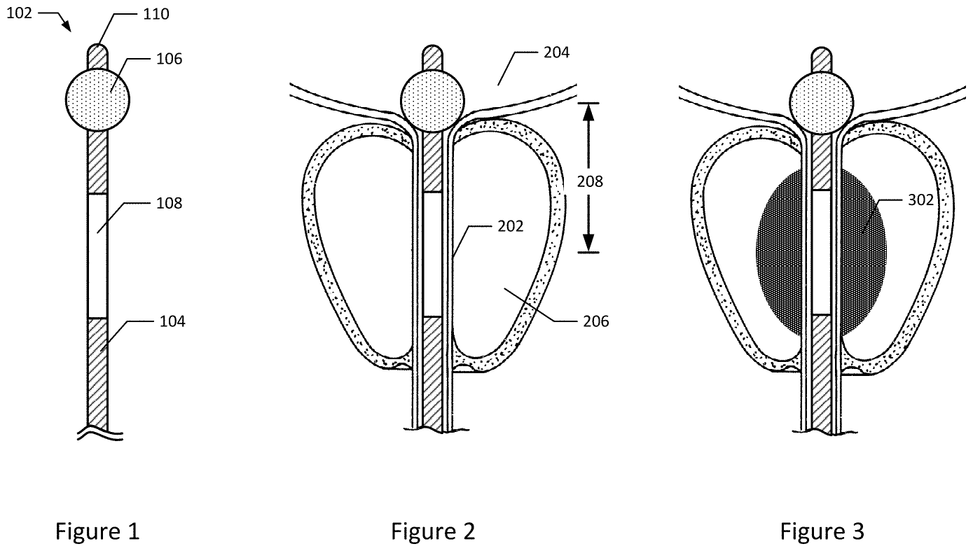

FIG. 1 shows an embodiment of the transurethral cryotherapy system. Cryotherapy catheter, or probe, 102 includes shaft 104, retention member or balloon 106, and cryotherapy section 108. Distal end 110 of the catheter is shown while the proximal end is not shown.

FIG. 2 shows an embodiment of the TC system in place in a patient. The TC catheter is inserted through urethra 202 until the distal tip, and the retention balloon, of the catheter is inside bladder 204. The retention balloon is deflated during insertion and is inflated after it is within the bladder, to anchor the catheter within the bladder. The catheter may be positioned so that the retention balloon sits snuggly at the junction of the bladder and the urethra, to aid in position the cryotherapy section of the catheter within prostate 206. Distance 208 between the retention balloon and the center of the cryotherapy section may vary among patients. As a result, TC catheters may come with different lengths 208 depending on the anatomy of the patient. Alternatively, distance 208 may be adjustable on a single TC catheter by sliding an outer shaft which includes the cryotherapy section with respect to an inner shaft to which the balloon is fixed. The TC catheter may be used to center the cryotherapy section within the prostate. Alternatively, the cryotherapy section may be positioned at a specific location within the prostate, which may not be the center. Distance 208 may be from about 3 mm to about 250 mm. Alternatively, distance 208 may be from about 20 mm to about 60 mm.

The TC catheter may also have a urine drainage lumen to allow urine to drain from the bladder through the catheter, similar to a Foley catheter. The TC catheter may be sized similar to a Foley catheter, or around 16 French.

FIG. 3 shows cryo-affected zone 302. This represents the tissue, primarily within the prostate, which has been affected by the cryotherapy treatment. The bulk of this tissue will necrose and shrink, allowing the prostate to shrink overall thus relieving BPH symptoms. Alternatively, depending on the applied temperature, the tissue may undergo apoptosis instead of, or in addition to, necrosis. Apoptosis may be preferable to necrosis because of the reduced tissue inflammation.

Cryotherapy may be administered using a cooling fluid, or cryogen, such as Nitrogen, Nitrogen Oxide, Carbon Dioxide, argon, air, or any other suitable cryogens. The coolant may be delivered to a substance, such as a metal such as copper, to cool the substance which in turn cools the tissue, either directly or through an intermediate zone of fluid, liquid, gas, air and/or tissue. Alternatively, the cryogen may be sprayed, vaporized, or administered from openings in the shaft of the TC catheter, directly to the tissue or to an intermediate zone near the tissue. The TC catheter is designed such that only a specific section, the cryotherapy section, delivers the cryotherapy. This protects the tissue that does not require treatment. The cryotherapy section is generally aligned with the prostate to help deliver therapy.

Temperature sensors may be placed at, near or distanced from the cryotherapy section of the TC catheter to determine the temperature of the treatment, the treated tissue, the nearby tissue and/or the untreated tissue. This temperature data may be used by a controller to control the treatment temperature to allow proper treatment, while minimizing damage to nearby tissue.

The cryo-affected zone may be controlled, both in size and shape, by the configuration of the cryotherapy section of the TC catheter. Several embodiments are included here to achieve different cryo-affected zone shapes/sizes.

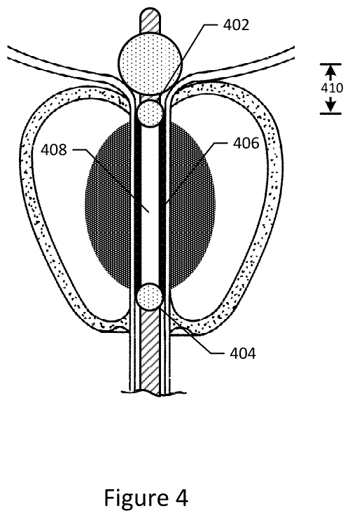

The embodiment of the TC catheter system shown in FIG. 4 includes a heating or insulation area to protect the urethra. The shape of the cryo-affected zone in this case may be more toroidal in shape rather than egg or cylindrical shaped. Heating or insulation zone 406 is a physical space between the urethra and the cryotherapy section 408. Section 408 may be smaller (or larger) in diameter than the rest of the catheter shaft. Heating zone 406 may be contained on one end by distal sealing balloon 402 and the other end by distal sealing balloon 404. Other sealing methods may be used as well. The retention balloon may serve as the distal sealing balloon. Heating zone 406 may be electrical heating, conductive heating, convective heating, or radiative heating, through the use of a heating element, a gas(es), liquid(s), or solid(s). Heating zone 406 may be enclosed in a balloon, in which case the sealing balloons may not be necessary. Heating zone 406 may cover the catheter shaft 360 degrees around or may cover only part of the catheter shaft radially. Heating zone 406 may not be actively heated, but may serve simply as passive thermal insulation. The geometry of this insulation can help control the size and shape of the cryo-affected zone to optimize for the clinical application or anatomy. Distance 410, the distance between the retention balloon and the most distal end of the cooling and heating/insulating area, may be controlled on the TC catheter to fit various patient and disease anatomies.

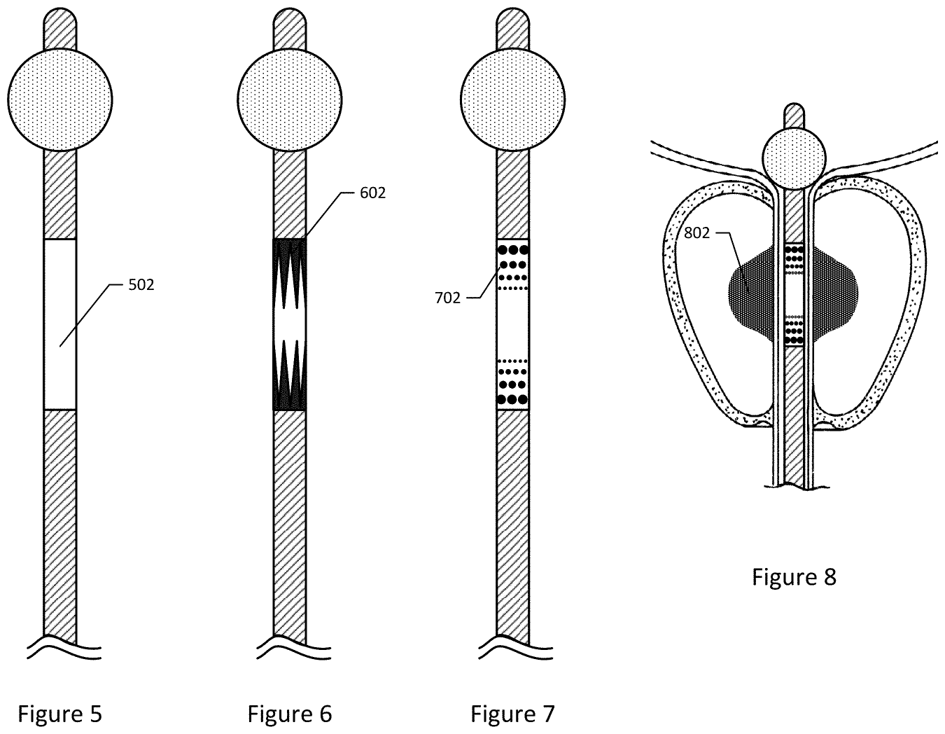

FIG. 5 shows a TC catheter without any insulation on cryotherapy section 502. FIG. 6 shows a TC catheter with variable insulation 602 on the cryotherapy section. FIG. 7 shows a TC catheter with variable insulation 702 on the cryotherapy section. The types of insulation shown in FIGS. 5-7 focus the cryotherapy toward the middle of the cryotherapy section. Many different shapes/insulation configurations are envisioned. By changing the insulation on the cryotherapy section, the cryo-affected zone 802 in FIG. 8, can be controlled.

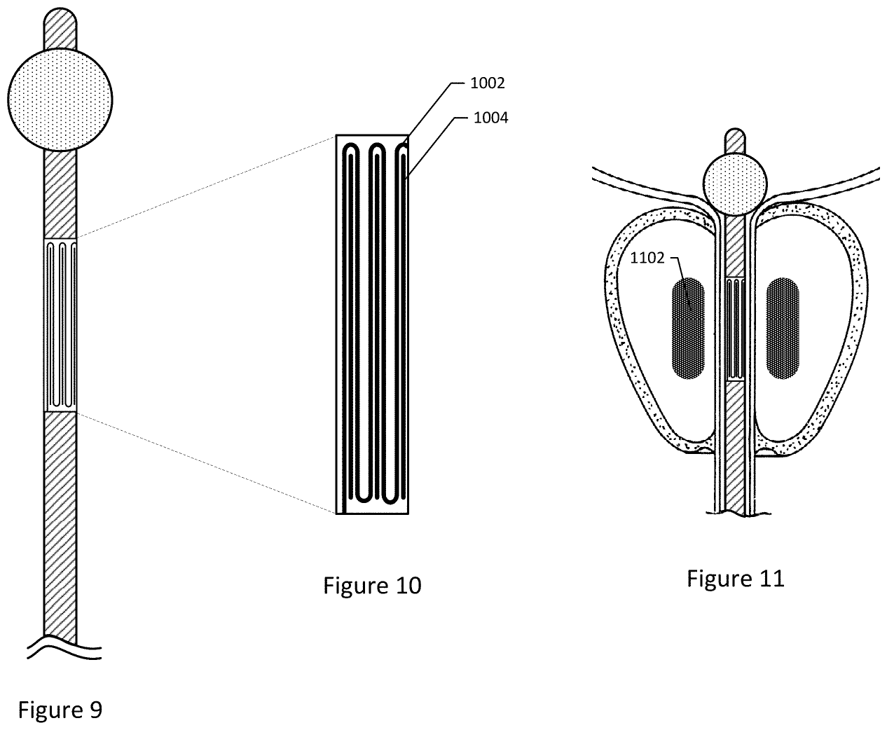

FIG. 9 shows a TC catheter with both heating and cooling elements in the cryotherapy section. FIG. 10 shows the cryotherapy section of FIG. 9 in detail. Heating element 1002 and cooling element 1004 may be intermingled, as shown here, or in separate sections. Again, the shape of the cryo-affected area can be controlled by the configuration of the cryotherapy section in this way.

FIG. 11 shows toroidal cryo-affected area 1102 caused by the TC catheter shown in FIG. 9.

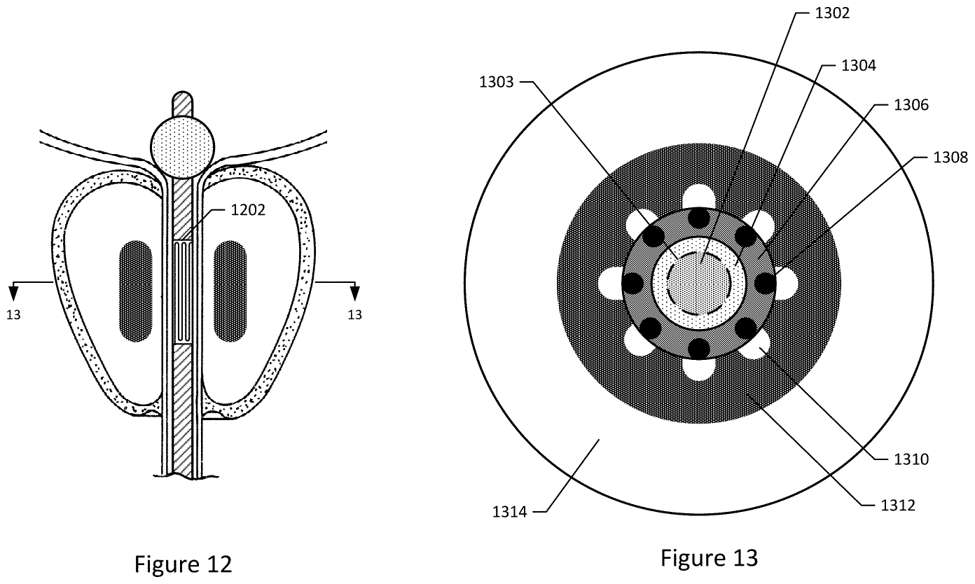

FIG. 12 shows another embodiment of a TC catheter with both heating and cooling elements in cryotherapy section 1202. A cross section of FIG. 12 is shown in FIG. 13. Here cryogen fluid 1302 flows through a lumen with openings 1303. This creates cryogen gas in lumen 1304. The cryogen gas cools cooling element 1306, which may be made out of any thermally conductive material such as metal, such as copper. Alternatively, cryogen fluid 1302 may be in direct contact with cooling element 1306. Warming elements 1308 warm the urethra to help prevent damage to the urethra. Cryo-affected zone 1312 includes protected areas 1310 of prostate 1314. The urethra may fall within protected areas 1310. In this way, the urethra can be protected somewhat from the cryotherapy.

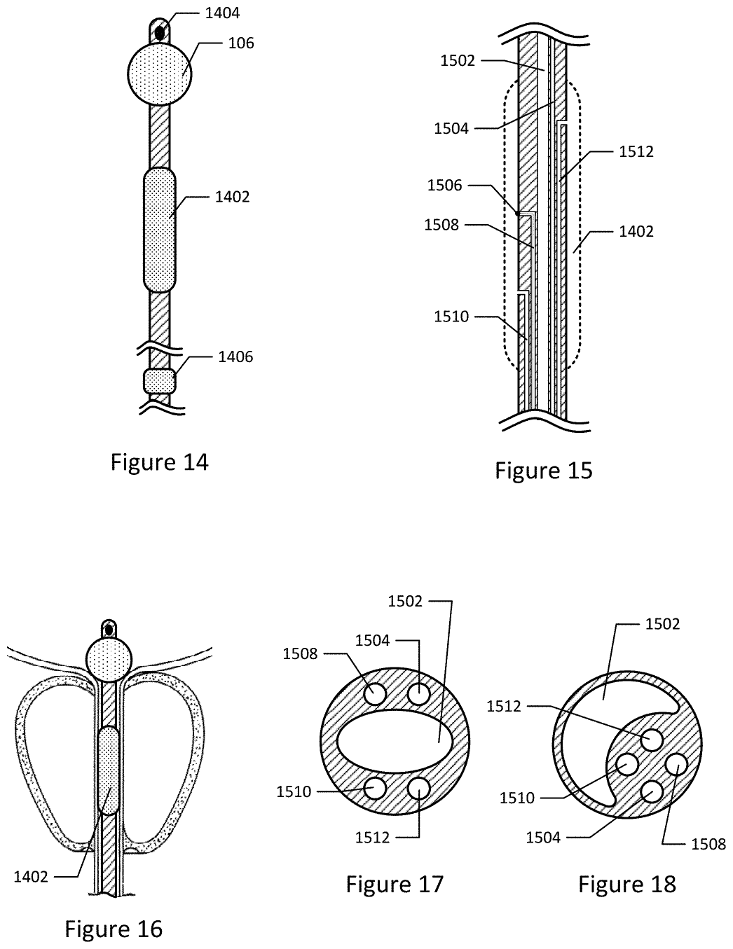

FIG. 14 shows an embodiment of the TC catheter including retention member or balloon 106, and cryotherapy balloon 1402. The distal end of the catheter includes opening or openings 1404 which are in fluid communication with an inner lumen, such as a urine lumen. Optional proximal stopper 1406 is also shown and can be used to maintain the position of the catheter within the urethra, i.e., preventing the catheter from migrating into the bladder. Stopper 1406 may be a balloon, a sliding component with a friction or other locking mechanism, or any other suitable mechanism. The distance between stopper 1406 and cryotherapy balloon 1402 or retention balloon 106 may be adjustable to accommodate different anatomies, including both male and female patients.

This embodiment uses a cryogen fluid, such as a cryogen gas, circulating within cryotherapy balloon 1402 to cause apoptosis or necrosis of targeted prostate tissue/cells. An example of a cryogen gas is argon gas. Other gases, such as air, may be used. Possible coolants/cryogens are listed in the table below:

| Coolant | Trade or | |
|---|---|---|
| Name | Common Name | CAS Name |
| R-717 | Ammonia | Ammonia |
| R-744 | CO2 | Carbon dioxide |
| R-290 | Propane | Propane |
| R-600a | Isobutane | Isobutane |
| R-170 | Ethane | Ethane |
| R-601 | Pentane | Pentane |
| R-161 | HFC-161 | Fluoroethane |
| R-123 | HCFC-123 | 2,2-Dichloro-1,1,1-trifluoroethane |
| R-225ca | HCFC-225ca | 3,3-Dichloro-1,1,1,2,2- |
| | | pentafluoropropane |
| R-152a | HFC-152a | 1,1-Difluoroethane |
| R-225cb | HCFC-225cb | 1,3-Dichloro-1,1,2,2,3- |
| | | pentafluoropropane |
| R-124 | HCFC-124 | 2-Chloro-1,1,1,2-tetrafluoroethane |
| R-32 | HFC-32 | Difluoromethane |
| R-141b | HCFC-141b | 1,1-Dichloro-1-fluoroethane |
| R-365mfc | HFC-365mfc | 1,1,1,3,3-Pentafluorobutane |
| R-245fa | HFC-245fa | 1,1,1,3,3-Pentafluoropropane |
| R-416A | | R-134a/R-124/R-600 (59/39.5/1.5) |
| R-401A | MP39 | R-22/R-152a/R-124 (53/13/34) |
| R-401B | MP66 | R-22/R-152a/R-124 (61/11/28) |
| R-414IB | Hot Shot | (50/39/1.5/9.5) |
| R-449 | Opteon XP40 | R-32/R-125/R-1234yf/R-134a |
| | | (24.3/24.7/25.3/25.7) |
| R-134a | HFC-134a | 1,1,1,2-Tetrafluoroethane |
| R-414A | GHX4 | R-22/R-124/R-600a/R-142b |
| | | (51/28.5/4.0/16.5) |
| R-409A | FX-56 | R-22/R-124/R-142b (60/25/15) |
| R-4310mee | HFC-43-10mee, | decafluoropentane |
| | HFC-4310mee, | |
| | R-43-10mee | |
| R-407C | | R-32/R-125/R-134a (23/25/52) |
| R-437A | MO49 Plus | R-32/R-125/R-600a/R-601 |
| | | (78.5/19.5/1.4/0.6) |
| R-22 | HCFC-22, Freon | Chlorodifluoromethane |
| R-407F | | R-134a/R-125/R-32 (40/30/30) |
| R-406A | | R-22/R-600a/R-142b (55/04/41) |
| R-413A | MO49 | R-218/R-134a/R-600a (9/88/3) |
| R-410A | Puron, AZ-20 | R-32/R-125 (50/50) |
| R-407A | KLEA 60 | R-32/R-125/R-134a (20/40/40) |
| R-427A | | R-32/R-125/R-143a/R-134a |
| | | (15/25/10/50) |
| R-438A | MO99 | R-125/R-134a/R-32/R-600a |
| | | (45/44.2/8.5/2.3) |
| R-423A | 39TC | R-134a/R-227ea (52.5/47.5) |
| R-142b | HCFC-142b | 1-Chloro-1,1-difluoroethane |
| R-417A | MO59, NU22 | R-125/R-134a/R-600 (46.6/50.0/3.4) |
| R-402B | HP-81 | R-125/R-290/R-22 (38/2/60) |
| R-424A | RS-44 | R-125/R-134a/R-600a/R-600/R-601a |
| | | (50.5/47/.9/1/.6) |
| R-422B | NU-22B | R-125/R-134a/R-600a (55/42/3) |
| R-421A | | R-125/R-134a (58/42) |
| R-422D | MO29 | R-125/R-134a/R-600a (65.1/31.5/3.4) |
| R-402A | HP-80 | R-125/R-290/R-22 (60/2/38) |
| R-407B | | R-32/R-125/R-134a (10/70/20) |
| R-422C | One Shot | R-125/R-134a/R-600a (82/15/3) |
| R-422A | | R-125/R-134a/R-600a (85.1/11.5/3.4) |
| R-227ea | HFC-227ea | 1,1,1,2,3,3,3-Heptafluoropropane |
| R-408A | FX-10 | R-125/R-143a/R-22 (7/46/47) |
| R-125 | HFC-125 | Pentafluoroethane |
| Isceon | | R-125/R-218/R-290 (86/9/5) |
| MO89 | | |
| R-404A | HP-62 | R-125/R-143a/R-134a (44/52/4) |
| R-507 | AZ-50 | R-125/R-143a (50/50) |
| R-403B | | R-290/R-22/R-218 (5/56/39) |
| R-143a | HFC-143a | 1,1,1-Trifluoroethane |
| R-502 | | R-22/R-115 (48.8/51.2) |
| R-11 | CFC-11 | Trichlorofluoromethane |
| R-113 | CFC-113 | 1,1,2-Trichlorotrifluoroethane |
| EP-88 | | |
| R-13b1 | Halon 1301 | Bromotrifluoromethane |
| R-115 | CFC-115 | Chloropentafluoroethane |
| R-14 | PFC-14, CF4 | Tetrafluoromethane |
| R-500 | | R-12/R-152a (73.8/26.2) |
| R-218 | PFC-218 | Octafluoropropane |
| R-236fa | HFC-236fa | 1,1,1,3,3,3-Hexafluoropropane |
| R-114 | CFC-114 | 1,2-Dichlorotetrafluoroethane |
| R-12 | CFC-12 | Dichlorodifluoromethane |
| R-116 | PFC-116 | Hexafluoroethane |
| R-508B | | R-23/R-116 (46/54) |
| R-13 | CFC-13 | Chlorotrifluoromethane |
| R-503 | | R-23/R-13 (40.1/59.9) |
| R-23 | HFC-23 | Trifluoromethane |

In some embodiments, the coolant is a refrigerant, or a coolant that cools to a less cold temperature and may evaporate at atmospheric pressure, and be liquefied at a lower pressure, for example, coolants with a boiling point above −180° C. For example, refrigerants in the R400 series (naming developed by DuPont® Corporation), which are generally zeotropic blends, may be used. For example, R410A may be used.

Cryotherapy balloon 1402 may have a radius of about 1.0 cm and a length of about 4.0 cm when inflated.

FIG. 15 shows a cross section of the TC catheter shown in FIG. 14, including some of the possible lumens. For example, drainage, or urine lumen 1502 is shown, as well as the inflation/deflation lumen 1504 for retention balloon 106, temperature sensor or thermocouple 1506 which is connected to a controller via temperature lumen 1508. The temperature sensor is used to sense the temperature within cryotherapy balloon 1402 which is used to regulate the cryogen fluid/gas within cryotherapy balloon to achieve a desired temperature of the cryotherapy balloon, and ultimately of the prostate tissue. Also shown are cryogen inflow lumen 1510 and cryogen outflow lumen 1512, which are used to control the flow of cryogen to and from the cryotherapy balloon.

FIG. 16 shows the embodiment of the TC catheter shown in FIG. 14 in place in the urethra. In some embodiments, cryotherapy balloon 1402 inflates just enough to achieve contact with the urethral wall, without exerting undo pressure on the urethra/prostate. In some embodiments, a controlled force is exerted on the urethra/prostate via the cryotherapy balloon. The cryotherapy balloon may be made out of a compliant material or a non-compliant material, depending on the forces and conformity desired. Suitable materials include silicone, polyethylene, other polymers and other materials.

FIGS. 17 and 18 show possible cross sections of the embodiment shown in FIGS. 14-16. Drainage lumen 1502 may be located approximately in the center of the catheter, as shown in FIG. 17, or may be located toward the outside of the catheter, as shown in FIG. 18. Locating the urine lumen outside of the cryogen inflow lumen 1510 and cryogen outflow lumen 1512 may help insulate the cryogen lumens, both allowing the fluid to remain cold, and also minimizing any cooling affects along the shaft of the catheter outside of the balloon area.

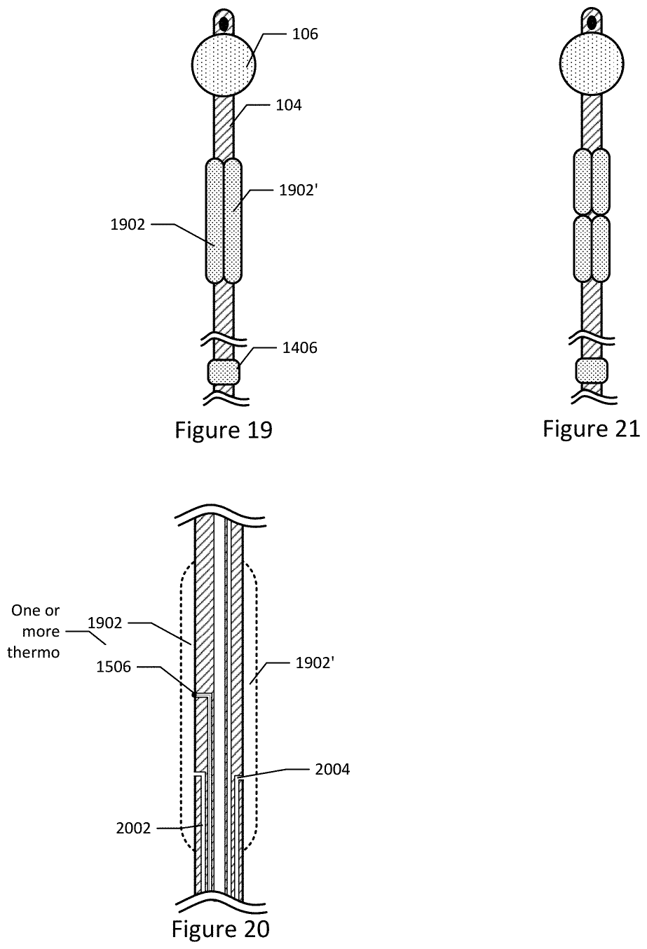

FIG. 19 shows an embodiment of the TC catheter with multiple balloons radially. Balloons 1902 and 1902′ are shown here. Two, three, or more balloons may be positioned radially around shaft 104. These balloons may serve as cryotherapy balloons or buffer balloons or both. A buffer balloon may be filled with air, water, or any other material designed to reduce the effect of the cryotherapy in that area. For example, if it is desired to only treat one area of the prostate, a balloon with cryogen may be placed closest to the treatment area, and a buffer balloon may be placed on the opposite side of the shaft to reduce the effect of the cryogen on the non-treatment side of the prostate. The cryogen filled balloon may expose a portion of the prostate to cryotherapy. For example, cryotherapy balloon(s) (or any other cryotherapy mechanism) may affect 0%-10% of the prostate radially. Alternatively, cryotherapy may affect 0%-25% of the prostate radially. Alternatively, cryotherapy may affect 0%-50% of the prostate radially. Alternatively, cryotherapy may affect 0%-75% of the prostate radially. A balloon may be a designated cryotherapy balloon or a designated buffer balloon, in which case the TC catheter may be placed in such a way that the therapy is directed to the desired portion of the prostate. Alternatively, each balloon may be used for either cryotherapy or buffering, in which case the therapy direction may be controlled by controlling which balloon is used for cryotherapy and which is used for buffering. The controller may also alternate between cryotherapy and buffering in any given balloon or area. The controller may control these functions.

FIG. 20 shows a cross section of an embodiment of the TC catheter with 2 radial balloons. Balloons 1902 and 1902′ are fed by lumens 2002 and 2004. Also shown is temperature sensor 1506. Each balloon may have a temperature sensor or a temperature sensor may only be present in cryotherapy balloons. A temperature sensor(s) may also or alternatively be placed along the shaft of the catheter to sense temperatures at different locations around and along the catheter length.

The cryogen may be introduced via cryogen inflow lumen 2002 and exit through a different cryogen exit lumen, or, the cryogen may enter and exit via the same lumen. In embodiments where the cryogen enter and exit a balloon via the same lumen, the cryogen may be introduced, and then actively removed by the controller at a preset time since introduction, or at a preset temperature since introduction. Alternatively, the cryogen may exit the lumen naturally when the pressure exceeds a cryogen exit pressure. This exit may be controlled by an active or passive valve.

FIG. 21 shows an embodiment of the TC catheter with multiple balloons radially and longitudinally. Similar to the embodiment shown in FIG. 19, the controller can control the delivery of cryogen and/or buffer to individual balloons to control the treatment and non-treatment areas of the prostate. Embodiments of the TC catheter may include one or more than one cryotherapy and/or buffer balloons or treatment areas. TC catheter embodiments may include multiple balloons radially and/or longitudinally.

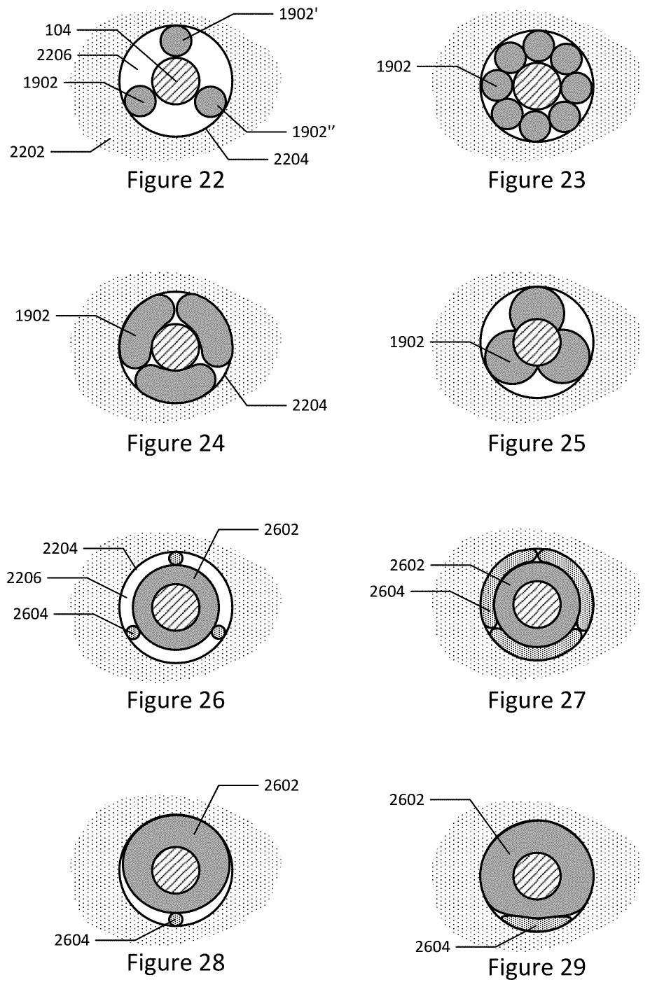

FIGS. 22-29 show cross sections of various TC catheter embodiments.

FIG. 22 shows an embodiment with 3 radial balloons, 1902, 1902′ and 1902″. These balloons may be filled with cryogen or buffer, depending on the desired treatment areas of prostate tissue 2202. The catheter may or may not include sheath 2204 which contains the various balloons and helps to hold them in position. Dead space 2206 may exist between the balloons depending on the configuration of the balloons. All or some of the balloons may be inflated with either cryogen or buffer. Some balloons may remain uninflated during treatment. Although 3 balloons are shown here, any number of balloons may be present, both radially and longitudinally. For example, FIG. 23 shows an embodiment with 8 radial balloons. Some embodiments may include only one balloon, which may be offset from the shaft. The balloons may be compliant or non-compliant.

FIG. 24 shows an embodiment of the TC catheter similar to that shown in FIG. 22, with 3 balloons. However in this embodiment, the balloons are not round when inflated, and are designed to each cover more of the radius of the urethra than the round balloons shown in FIG. 22. In other words, the catheter, when all balloons are inflated, may create less dead space than the embodiment shown in FIG. 22. The balloons in FIG. 24 may be enclosed in sheath 2204. As with any of the embodiments disclosed herein, each balloon may be inflated with cryogen or buffer, or may alternate between cryogen and buffer. Coolant and/or buffer may be pulsed through the balloon.

FIG. 25 shows another embodiment of the TC catheter with 3 balloons. In this embodiment, the balloons are attached to the shaft along its length so that they are semicircular when inflated.

FIG. 26 shows another embodiment of the TC catheter. In this embodiment, the cryotherapy balloon 2602 is essentially concentric with the shaft and is adjacent to, or surrounded by one or more than one external buffer balloon 2604. The buffer balloon(s) serve to shift the directionality of the cryotherapy away from or toward certain areas of the prostate. For example, one of the buffer balloons may be inflated, directing the cryotherapy in the opposite direction. Or two of the balloons may be inflated, to direct the cryotherapy in a different direction.

FIG. 27 shows an embodiment of the TC catheter similar to that shown in FIG. 26. The embodiment shown in FIG. 27 creates less dead space than that shown in FIG. 26.

FIGS. 28 and 29 show embodiments of the TC catheter with only one external buffer balloon. In these embodiments, the buffer balloon is used to direct the cryotherapy toward the prostate on the opposite side of the buffer balloon. In these embodiments, it is beneficial to identify the desired treatment area of the prostate before positioning the catheter so that the cryotherapy can be directed toward the treatment area. In embodiments with more than one buffer balloon, it is possible to insert the catheter without orienting it, and then to simply apply cryotherapy to the appropriate balloon(s) so that the target treatment area is treated. Different cryotherapy balloons may be marked so that the markings show up under ultrasound or other imaging and each balloon can be identified after insertion. The desired treatment area/balloons can then be entered into the controller to control treatment. In some embodiments, gravity may be used to direct the orientation of the catheter and/or the treatment.

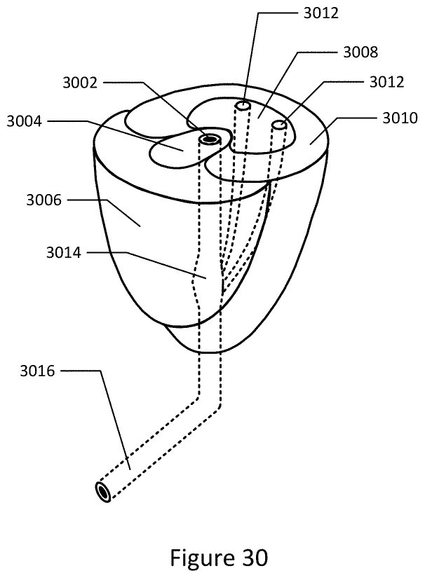

FIG. 30 shows the prostate anatomy. Shown are urethra 3002, various zones of the prostate, including transitional zone 3004, anterior region 3006, central zone 3008, peripheral zone 3010, as well as ejaculatory duct 3012, area of seminal colliculus 3014, and penile urethra 3016. Depending on the patient, the physician may want to treat certain area(s) and protect other area(s). In general, the physician will want to protect the ejaculatory duct and the area of seminal colliculus. The physician may want to focus the cryotherapy on any one or more of the central, peripheral, anterior or transitional zones. To do so, the TC catheter cryotherapy and buffer balloons may be arranged so that the cryotherapy is focused on the treatment area(s), and the buffer balloons are adjacent the area(s) that is to be protected. This may be done both radially and longitudinally. For example, the physician may want to target the upper transitional zone, avoiding the lower area of seminal colliculus and the central zone. Different balloons in different areas of the TC catheter may be inflated with cryogen and buffer to affect and protect the desired areas.

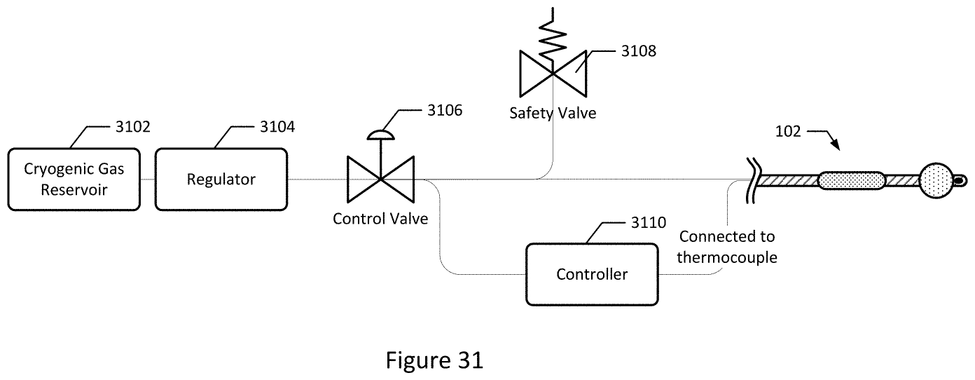

FIG. 31 shows an example of components of a controller of the TC System. Some or all of the components of the controller may be handheld or portable. Shown here is a replaceable canister or reservoir 3102 of cryogenic gas, such as argon, regulator 3104, control valve 3106 to adjust gas flow rate into and out of the catheter, safety valve 3108, and a controller component 3110, such as a PID controller, that controls and maintains the internal cryogen balloon temperature over time, as well as the balloon pressure, by controlling the inflow and outflow of the cryogen gas/fluid.

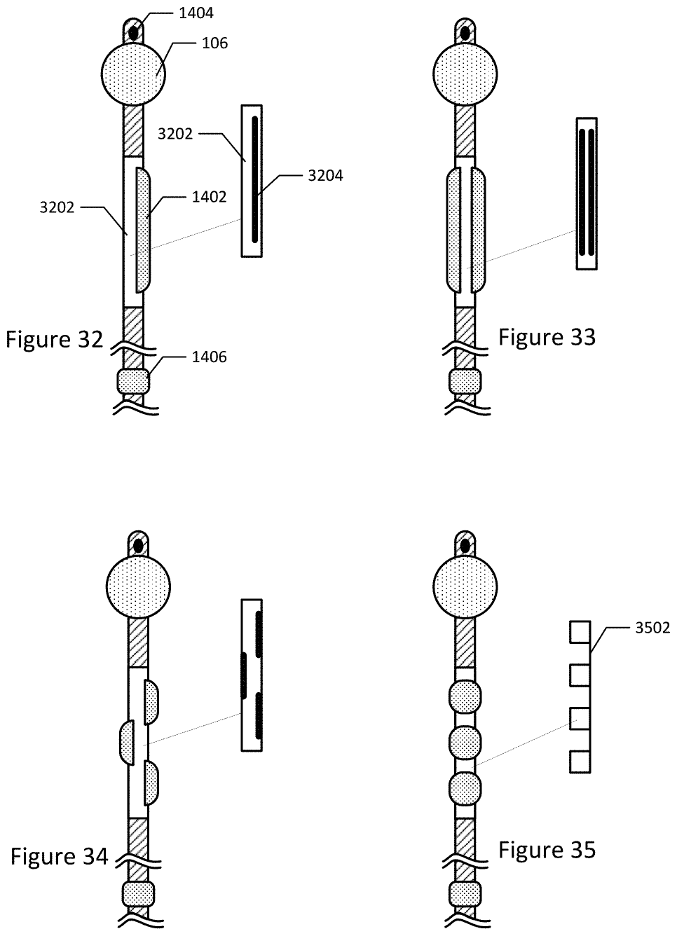

FIGS. 32-35 show embodiments of the TC system that allow for directional application of the therapy, including cryotherapy. These embodiments include sheath or casing 3202 which restricts all but one or more portions of cryotherapy balloon 1402. For example, FIG. 32 shows a cryotherapy catheter/probe/system where cryotherapy balloon 1402 is restricted by sheath 3202 which has opening 3204. The sheath is placed over the cryotherapy balloon area, either before the procedure, or during the procedure, to partially restrict the cryotherapy balloon from inflating/expanding in all directions. FIG. 32 shows a sheath with one longitudinal opening which allows the cryotherapy balloon to expand on only one side of the catheter. Because the cryogen is only substantially filling the cryotherapy balloon in this one area, treatment of the prostate is also limited to this one area. In this way, the cryotherapy can be directed toward specific areas of the prostate for therapy, while protecting other areas. The areas of the prostate in contact with the sheath, where the sheath fully covers the cryotherapy balloon, will not be exposed to as much cooling as the area of the prostate immediately adjacent the protruding cryotherapy balloon section, i.e. the portion of the cryotherapy balloon which exits through the sheath opening.

The sheath may be positioned onto the catheter before the procedure, depending on the anatomy. Different types of sheaths may be used depending on the prostate treatment area and area that is to be protected. In some embodiments, the sheath may be repositioned or replaced during the procedure. For example, in some embodiments the sheath may be connected to an elongated guide (not shown) that extends all or most of the length of the catheter so that the physician can move the sheath while the catheter is in place in the prostate.

FIG. 33 shows an embodiment of the TC system with a directional sheath with more than one elongated opening, which allows more than one lobe of the cryotherapy balloon to protrude.

FIG. 34 shows an embodiment of the TC system with more than one opening in the sheath, resulting in more than one opening radially and more than one opening longitudinally. The embodiments shown in FIGS. 32-34 show sheaths with openings that restrict the cryotherapy balloon to only a portion of the circumference of the catheter.

FIG. 35 shows an embodiment of the TC system where the sheath restricts the cryotherapy balloon longitudinally, but not radially. The sheath in this embodiment may be essentially one or more circumferential bands which may or may not be connected with retainer 3502. Retainer 3502 may be a wire, a thread or other element. In this embodiment, the retainer element does not substantially restrict the expansion of the cryotherapy balloon circumferentially, but in some embodiments the retainer may restrict the expansion.

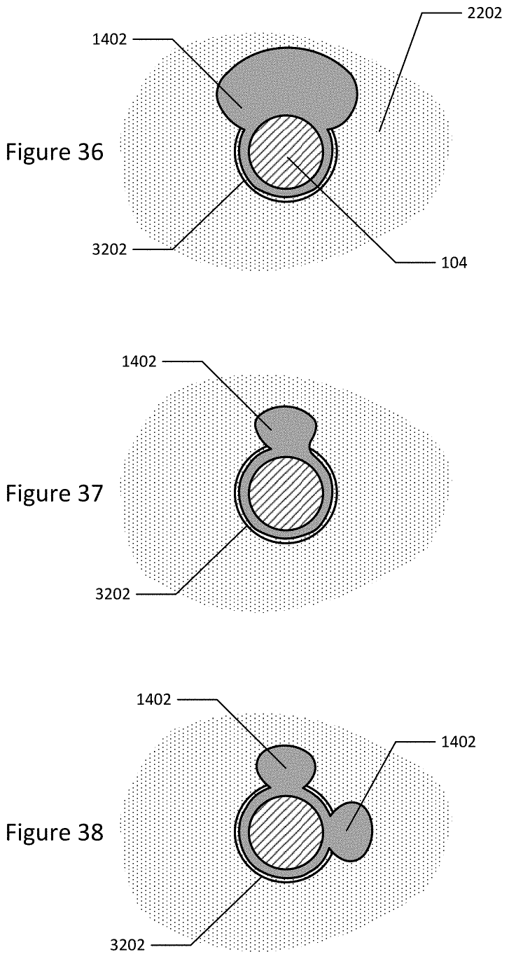

FIGS. 36 and 37 show cross-sectional views of some embodiments of the TC system which incorporate restrictive sheaths 3202. FIGS. 36 and 37 show embodiments with sheath openings that allow one lobe of the cryotherapy balloon to protrude circumferentially. For example, FIG. 37 may represent a cross section of the embodiment shown in FIG. 32. FIG. 36 shows a similar embodiment, although with a larger opening resulting in a larger protruding balloon lobe and a larger prostate treatment area circumferentially.

FIG. 38 shows an embodiment with a sheath that allows for two balloon lobes circumferentially.

Sheath 3202 may be made from a polymer, metal, silicone, or other suitable material.

Some embodiments of the sheath have markings or areas which are visible under visualization, such as ultrasound, so that the physician can identify the position of the openings of the sheath with respect to the prostate while it is in position. This allows the physician to target the therapy at the areas which need it while protecting the areas of the prostate that do not require therapy.

Some embodiments of the TC system may include a relatively compliant cryotherapy balloon. Some embodiments of the TC system may include a relatively non-compliant cryotherapy balloon. The sheaths used with relatively non-compliant balloons may require larger openings than those used with relatively compliant balloons, to allow enough of the balloon to protrude and effectively treat the prostate.

Although most embodiments disclosed herein refer to a system designed to treat the prostate, it is understood that many of the embodiments may be used for other applications. For example, any of the treatment, or balloon orienting embodiments may be used for applying temperature, pressure or other directed treatments to blood vessels or other organs. For example, embodiments with balloon restricting sheaths, such as those shown in FIGS. 32-38, may be used for angioplasty, or other balloon dilatation devices, to direct the pressure in certain areas of the anatomy, while protecting others. This may be useful in the areas of branched vessels, stent deployment, or other anatomical situations. Other applications might include strokes, etc.

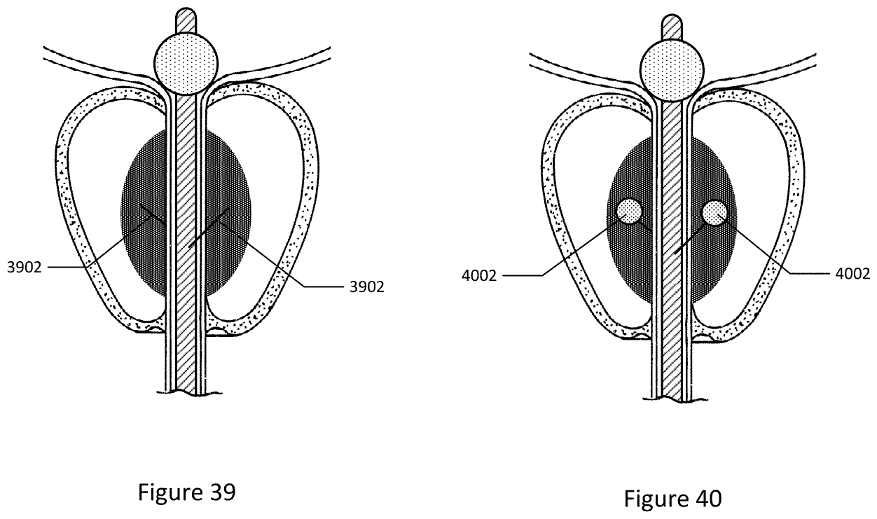

FIGS. 39 and 40 show embodiments of the TC system which incorporate one or more needles 3902. Needles may be deployed by the device to enter the prostate tissue by passing through the urethra wall into the prostate. Cryotherapy may be locally delivered to the prostate via one or more needles. The cryotherapy may be very precisely directed in this way.

FIG. 40 shows an embodiment of the TC system similar to that shown in FIG. 39.

The embodiment in FIG. 40 includes cryotherapy balloon(s) 4002 which may be inflated with cryogen within the prostate tissue.

Some embodiments of the TC System effectively debulk the prostate gland by causing cell apoptosis, which substantially avoids the side effects and inflammation of cell necrosis. This is done via the localized delivery of sublethal temperature cryogen. Cryogen temperatures may be 0 to −20° C. or alternatively, 0 to −40° C. or alternatively, 0 to −50° C. or alternatively, −10 to −20° C. or alternatively, −20 to −40° C. or alternatively, −20 to −50° C. More than one temperature may be used over time to affect the cells. Adipose cells exposed to these temperatures undergo apoptotic mediated cell death and are subsequently cleared by macrophages to reduce localized fat tissue. This treatment modality is advantageous in that apoptosis, or programmed cell death, does not induce a host immune response, therefore reducing the risk of chronic inflammation. Sublethal cryotherapy may also limit peripheral damage, preventing damage to peripheral nerves in the penis essential for the preservation of sexual function.

Embodiments of the TC catheter/system disclosed herein allow for uniform cryothermic delivery to both lateral lobes of the prostate through a single transurethral approach. The TC catheter may utilizes a low durometer polyurethane medical balloon inflated with a cryogen to widen the urethral lumen and deliver a uniform cryothermic dose to the prostate. By optimizing the balloon dimensions, balloon pressure, duration of cryotherapy administered, and the rate of cooling, the TC system can provide cryothermic dosing to the entire prostate through a single transurethral approach without the need for repositioning.

The temperature applied to the prostate may vary over time. For example, the TC system may first expose the prostate to a colder temperature, to cool the tissue quickly, then switch to a slightly less cold temperature, so that cell necrosis is limited. For example, the TC system may expose the prostate to a temperature around −50° C. for a period of time, then adjust the applied temperature to around −20° C. for a period of time to cause cell apoptosis. In some embodiments, the target temperature of the majority of the prostate cells is about −15° C. In some embodiments, the temperature of the majority of the prostate cells does not drop below (colder than) −20° C.

In some embodiments, the prostate cells are maintained at a temperature of about −10° C. to about −20° C. for a period of 30-60 minutes. In some embodiments, the prostate cells are maintained at a temperature of about −15° C. to about −20° C. for a period of 30-60 minutes. In some embodiments, the prostate cells are maintained at a temperature of about −10° C. to about −20° C. for a period of 0-30 minutes. In some embodiments, the prostate cells are maintained at a temperature of about −15° C. to about −20° C. for a period of 0-30 minutes. In some embodiments, the prostate cells are maintained at a temperature of about −10° C. to about −20° C. for a period of 60-120 minutes. In some embodiments, the prostate cells are maintained at a temperature of about −15° C. to about −20° C. for a period of 60-120 minutes.

Some embodiments of the TC system may cycle the temperature of device through higher and lower temperatures once or repeatedly over time.

Some embodiments of the TC system may follow the cooling period with a warming period, by infusing warm fluid, such as water, into the balloon or into the catheter. The timing and temperature of the cooling and/or warming periods may be precisely controlled so that the tissue exposure to cold is tightly controlled. The control of the cooling and/or warming periods' temperature and/or time may be controlled manually, or may be controlled automatically or may be controlled via a feedback mechanism, such as temperature sensors in the system.

Embodiments disclosed herein may or may not include a drainage or urine lumen.

Variables that are controlled for the cryotherapy include: min temperature (degrees C.), min temperature hold time (seconds), cooling rate (degrees C./second), thawing rate (degrees C./second), pressure, etc. The treatment can involve one cooling cycle or multiple cooling cycles. Min temperature can range from about 25C to about −40C. Alternatively, Min temperature can range from about 10C to about −10C. Alternatively the range can be between about 0C to about −20C. Hold time for a given cycle can range from about 5 seconds to about 60 minutes. Cooling and heating rates can range from about 1C/minute to about 500C/minute.

In some embodiments that use a coolant, the coolant may be cycled through a balloon while the balloon is positioned within the urethra in proximity to the prostate. The coolant may be introduced into a balloon, and after a period of time, vacuumed out using a pump or similar in communication with the controller. This vacuuming may be automatically performed by the controller or performed manually. There may be more than one cycle of introducing and removing coolant from a balloon during a procedure. Alternatively, a balloon may have more than one entry point so that the coolant can by cycled through either in boluses, or continually or semi-continually.

Cryotherapy has advantages over radio frequency or microwave ablation technologies. These technologies rely on electromagnetic radiation to excite tissues and elevate their temperature and cause an ablation. Polar molecules, such as water, are particularly excited by electromagnetic radiation such as microwaves. Stromal and epithelial tissue has little water content compared to muscle tissue and therefore it is more challenging to ablate fatty tissues with electromagnetic radiation. Since cryoablation causes apoptosis through thermal shock or modification, it is less sensitive to water content in tissues and can have a meaningful and controllable impact on fatty tissues.

Post procedure the stromal and epithelial cells will be ablated and slowly removed and reduced in size by the body, reducing impingement of the urethra. Benefits include increased urinary flow rates, frequency of nocturia, frequency of bladder emptying and post void urine residual.

The energy to heat the heating elements of the TC catheter and/or to cool the cooling elements of the TC catheter are supplied by an energy applicator which may be part of the controller. The controller may also include temperature sensing elements, temperature control system, balloon pressure sensing elements, etc.

Some embodiments of the transurethral cryotherapy system for treatment of benign prostatic hyperplasia include an energy applicator for applying a cooling temperature to urethra tissue adjacent to the applicator and to prostate tissue which surrounds the urethra tissue and extends radially outwardly therefrom; and a temperature control system for sensing the temperature of the urethra tissue and decreasing the cooling temperature applied by the applicator based on the sensed temperature so that the urethra tissue temperature decreases in increments, wherein the temperature control system includes a temperature sensor disposed near the urethra tissue for sensing the temperature of the urethra tissue and producing a temperature output signal indicative of the temperature, a controller coupled to the temperature sensor, receiving the temperature output signal, and producing a programmed temperature control signal in response to the temperature output signal, and a cooling temperature source coupled to the controller, receiving the temperature control signal, and regulating the energy applied by the applicator based on the temperature control signal such that the applicator simultaneously maintains the urethra tissue at a set temperature for a set amount of time required for treatment of benign prostatic hyperplasia.

Example of Data Processing System

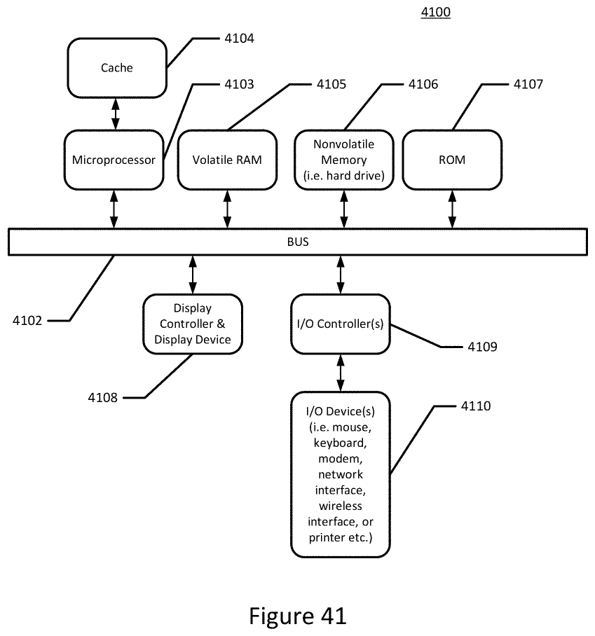

FIG. 41 is a block diagram of a data processing system, which may be used with any embodiment of the invention. For example, the system 4100 may be used as part of the controller/processor. Note that while FIG. 41 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 41, the computer system 4100, which is a form of a data processing system, includes a bus or interconnect 4102 which is coupled to one or more microprocessors 4103 and a ROM 4107, a volatile RAM 4105, and a non-volatile memory 4106. The microprocessor 4103 is coupled to cache memory 4104. The bus 4102 interconnects these various components together and also interconnects these components 4103, 4107, 4105, and 4106 to a display controller and display device 4108, as well as to input/output (I/O) devices 4110, which may be mice, keyboards, modems, network interfaces, printers, and other devices which are well-known in the art.

Typically, the input/output devices 4110 are coupled to the system through input/output controllers 4109. The volatile RAM 4105 is typically implemented as dynamic RAM (DRAM) which requires power continuously in order to refresh or maintain the data in the memory. The non-volatile memory 4106 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 41 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 4102 may include one or more buses connected to each other through various bridges, controllers, and/or adapters, as is well-known in the art. In one embodiment, the I/O controller 4109 includes a USB (Universal Serial Bus) adapter for controlling USB peripherals. Alternatively, I/O controller 4109 may include IEEE-1394 adapter, also known as FireWire adapter, for controlling FireWire devices, SPI (serial peripheral interface), I2C (inter-integrated circuit) or UART (universal asynchronous receiver/transmitter), or any other suitable technology. Wireless communication protocols may include Wi-Fi, Bluetooth, ZigBee, near-field, IR (infrared), cellular and other protocols.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments of the systems and/or devices and/or methods enclosed herein may be used for other clinical applications also, including treating the sinuses, the esophogus, the intestines, the gall bladder, etc.

What is claimed is:

1. A cryotherapy apparatus, comprising:
a catheter which is sized for placement within a body lumen;
at least one balloon positioned along a length of the catheter and defining a contact region for thermally contacting a portion of the body lumen which is in proximity to tissue to be treated;
a reservoir in fluid communication with the at least one balloon through at least one lumen defined through the length of the catheter,
wherein the at least one balloon is configured to receive a refrigerant from the reservoir introduced through the at least one lumen until the contact region is at least partially expanded, the catheter being adjustable to selectively align the contact region against the portion of the body lumen to preferentially cool the portion relative to a remainder of the body lumen surrounding the portion.

2. The apparatus of claim 1 wherein the refrigerant has a boiling point above −180° C.

3. The apparatus of claim 1 wherein the catheter is sized for transurethral insertion.

4. The apparatus of claim 1 wherein the refrigerant has a temperature sufficient to treat benign prostate hyperplasia in proximity to the body lumen when the refrigerant is introduced into the at least one balloon.

5. The apparatus of claim 1 wherein the at least one balloon is positionable against the body lumen such that the refrigerant is introducible into the at least one balloon to selectively cool the portion of the body lumen via conductive cooling.

6. The apparatus of claim 1 further comprising an insulating component positioned along the catheter for insulating portions of the body lumen.

7. The apparatus of claim 1 further comprising two or more expandable balloons which are positionable along the catheter.

8. The apparatus of claim 7 wherein the two or more expandable balloons are expandable independently from one another.

9. The apparatus of claim 7 wherein the two or more expandable balloons are positionable along the catheter to selectively press the contact region against the body lumen.

10. The apparatus of claim 7 wherein the refrigerant is selectively introduced into one of the two or more expandable balloons to cool the portion of the body lumen.

11. The apparatus of claim 1 further comprising a sheath which shields a portion of the at least one balloon.

12. The apparatus of claim 1 further comprising one or more warming areas positioned along the catheter for contact against the body lumen.

13. A method of applying a cryotherapy treatment, comprising:
advancing a catheter within a body lumen such that at least one balloon positioned along a length of the catheter is placed near or at a portion of the body lumen which is in proximity to tissue to be treated;
adjusting a position of the catheter to align a contact region defined along the at least one balloon into contact against the portion of the body lumen;
initiating a refrigerant to flow through at least one lumen defined through the length of the catheter and into the at least one balloon such that the contact region is at least partially expanded into thermal contact against the portion of the body lumen; and
applying the refrigerant through the contact region until the tissue to be treated is preferentially cooled through the portion of the body lumen relative to a remainder of the body lumen surrounding the portion.

14. The method of claim 13 wherein the refrigerant has a boiling point above −180° C.

15. The method of claim 13 wherein advancing the catheter comprises advancing the catheter transurethrally.

16. The method of claim 15 wherein applying the refrigerant comprises applying the refrigerant to treat benign prostate hyperplasia.

17. The method of claim 13 wherein applying the refrigerant comprises conductively cooling the portion of the body lumen.

18. The method of claim 13 further comprising insulating portions of the body lumen not subject to treatment.

19. The method of claim 13 wherein initiating the refrigerant comprises expanding two or more balloons positioned along the catheter to urge the contact region against the portion of the body lumen.

20. The method of claim 19 wherein initiating the refrigerant comprises selectively introducing the refrigerant into the two or more balloons to cool the portion of the body lumen.

21. The method of claim 19 further comprising positioning a sheath relative to the at least one balloon prior to applying the refrigerant.

22. The method of claim 13 further comprising warming one or more elements positioned along the catheter in contact against the body lumen.
